(19) United States
(12) Patent Application Publication
(10) Pub. No.: US 2004/0109880 A1
(43) Pub. Date: Jun. 10, 2004

(54) Cosmetic and/or pharmaceutical preparations

(76) Inventors: Gilles Pauly, Nancy (FR); Philippe Moser, Essey-Les-Nancy (FR); Louis Danoux, Saulxures Les Nancy (FR); Olga Freis, Seichamps (FR); Florence Henry, Villers-Les-Nancy (FR); Muriel Pauly-Florentiny, Nancy (FR); Anne Guezennec, Croissy Sur Seine (FR); Joelle Guesnet, Bures Sur Yvette (FR); Philippe Moussou, Nancy (FR)

Correspondence Address: COGNIS CORPORATION, PATENT DEPARTMENT, 300 BROOKSIDE AVENUE, AMBLER, PA, 19002, US

(21) Appl. No.: 10/250,814

(22) PCT Filed: Jan. 5, 2002
(86) PCT No.: PCT/EP02/00055

(30) Foreign Application Priority Data
Jan. 15, 2001 (FR) 0100492

(51) Int. Cl. A61K 38/17; A61K 6/00; A61K 7/04

(52) U.S. Cl. 424/401; 514/12

(57) ABSTRACT

A cosmetic or pharmaceutical composition containing an active ingredient selected from the group consisting of late embryogenesis abundant proteins, heat shock proteins, and mixtures thereof.

### FIELD OF THE INVENTION

This invention relates generally to the field of cosmetics and, more particularly, to preparations with an effective content of special vegetable and/or microbial proteins and to the use of the proteins for the production of the preparations.

### PRIOR ART

A key reason for the ageing of skin is the loss of water from the upper layers of the epidermis and the wrinkling associated therewith. Accordingly, one of the ways cosmetic chemists seek to counter this phenomenon is to provide active substances which counteract environmental stress and dehydration and/or which have a protective function so that the cells are fortified in their ongoing struggle against environmental poisons. To this end, occasionally unusual pathways have to be followed to find a solution. Thus, it may be appropriate to gather important information from the knowledge with which nature provides us and to apply it to meet particular needs.

In nature, two families of proteins inter alia play an important part in reacting to various environmental influences and stress factors. These two protein families which are involved in the resistance mechanism are the late embryogenic abundant proteins, also known as LEA proteins, which are mainly found in the plant world, and the LEA-like proteins, the heat shock proteins (HSPs), which are found in plants and microorganisms and also in animals and man. LEA proteins can be extracted, for example, from so-called resurrection plants or from seeds or scions of barley, wheat, corn, rise, peas cowpeas, soya, onions, tomatoes, cotton, radishes, cucumbers and firs (Close T. J. et al. (1993), Plant Molecular Biology, 23, 279-286; Walters C. et al. (1997), Seed Science Research, 7, 125-134; Russouw P. S. et al. (1995), Seed Science Research, 5, 137-144). The molecular weights of the proteins are in the range from 11 to 150 kDa.

In the desert regions and arid zones of Africa, Asia and America, a number of plant families have developed a remarkable tolerance to drought which enables them to withstand up to 98% dehydration over a period of one year without damage and thereafter to regenerate themselves completely and to form flowers within 24 hours of the first monsoon rains. These poikilohydric representatives are known collectively as resurrection plants and include mosses, lichens and ferns and a number of flowering plants (angiospermae) of which studies have shown that the anatomical, biochemical and physiological adaptation is attributable to the genome.

During the drought phase, the plants are exposed to two different stresses, i.e. on the one hand mechanical stress and, on the other hand, oxidative stress. Resurrection plants have a number of ways of avoiding mechanical stress, of which shrinkage and the sharing of vacuoles to reduce stress on the plasma membrane are generally widespread. Other effects include the increased incorporation of xyloglucans and methylesters of pectin in the cell wall and the accumulation of osmolytes or osmoregulating molecules (for example sucrose, mannitol, D-ononitol, trehaloses, fructans, amino acids, etc.), so that the cell wall is strengthened and the production of toxic metabolites during dehydration is suppressed.

In addition, the interruption of cell respiration and photosynthesis during the drought phase leads to the formation of free radicals which are capable of damaging proteins, fats and nucleic acids. To prevent this, pigments of the anthocyan type and special enzymes are increasingly encountered in the cells, including for example superoxide dismutase, gutathione reductase and ascorbate peroxidase, which engage in the oxidative metabolism and are known as natural radical trappers.

The molecular fundamentals of tolerance to drought have not yet been fully elucidated. However, according to investigations conducted by D. Bartels at Bonn University, it seems clear that plant hormones, such as abscisic acid (ABA) for example, induce tolerance to drought. Since those investigations, a number of genes involved both in the process of desiccation and in rehydration have also been isolated. It was surprisingly found that those genes are homologous to genes that are also found in embryos of ripening seeds. For example, the gene dsp-22 (desiccation stress protein) is activated in the event of desiccation and stimulates the formation of a 21 KDa protein which accumulates in the chloroplasts [cf. D. Bartels et al., EMBO Journal, 11(8), 2771 (1992)]. In addition, changes in the metabolism of sugars are of importance. For example, the leaves of unstressed plants show high concentrations of the unusual sugar 2-octulose which is converted during desiccation into sucrose and appears to perform a protective function in the process. The process is reversible on rehydration. Reference is also made in this connection to International Patent Application WO 97/42327 (University of Mexico) which reports on the isolation of a gene from the resurrection plant Selaginella lepidophylla which produces the sugar trehalose-6-phosphate.

Accordingly, the problem addressed by the present invention was to provide new active substances with which, in general terms, the skin and hair could be protected from environmental influences and, more particularly, the skin could be prevented from drying out. In addition, the skin and hair would be afforded additional protection against osmotic, mechanical and temperature-induced shock and stress.

### DESCRIPTION OF THE INVENTION

The present invention relates to cosmetic and/or pharmaceutical preparations containing late embryogenesis abundant (LEA) proteins and/or LEA-like proteins.

It has surprisingly been found that the vegetable or microbial proteins mentioned satisfy the problem stated above in excellent fashion.

Late Embryogenesis Abundant (LEA) Proteins

The name late embryogenesis abundant proteins stems from the observation that certain hydrophilic proteins form or accumulate in late stages of the embryogenesis of seeds although the name is not confined to proteins which are formed in the course of plant development. In fact, these substances can be found in all parts of the growing or fully grown plant, i.e. for example in roots, stalks and leaves. LEA proteins, which are surface-active polypeptides with around 50 to 1,000 and preferably 100 to 600 monomer units, represent important constituents of fractions which are found mainly in the cell core and in the cytoplasm and which can be isolated above all by extraction of so-called resurrection plants and also of wheat gluten or other vegetable protein sources and fungi, algae and lichens. An overview of this subject by Wolters et al. can be found in Amer. Soc. Plant Physiol. 114(S), 113 (1997).

Resurrection plants are not a coherent group but can be found in very different plant families, among which the families of the Poacea, Scrophulariacea, Myrothamnacea and/or Velloziacea are mentioned above all. The most important representatives of the Poaceae include the genus Spirobolus, for example a grass which grows to a height of 60 to 120 cm and develops pink-colored flowers. It occurs above all on the American continent, especially in Costa Rica, where the species Spirobolus cubensis, Spirobolus indicus, Spirobolus heterotepsis, Spirobolus capillaris, Spirobolus flexuosus, Spirobolus cryptandrus and Spirobolus airoides can be found. A particularly important example of a resurrection plant from the family of the Scrophulariaceae is the genus Craterostigma, more particularly the species Craterostigma plantigineum. From the family of the Myrothamnaceae, mention is made above all of Myrothamnus niedenzu and Myrothamnus flabellifolia which was described for the first time in 1891 by Engler and Pranti. This plant is a flat shrub which does not shed its leaves in the dry winter months, but applies them flat against the branches and comes back to life with the first summer rains. Key constituents of the extracts of its leaves are arbutin, anthocyans, polysaccharides (sucrose, glucose, trehalose, fructose, glucosyl-9-glycerol) and phytohormones (for example abscisic acid); terpenes such as, for example, carvones and perillic alcohol can also be found. Like octulose, arbutin also plays an important, albeit different, role in resistance to drought because, as a hydroquinone source, it prevents the peroxidation of unsaturated lipids in the cell membranes. Typical examples of resurrection plants from the Velloziacea family are the representatives of the genus Xerophyta, such as for example the Xerophyta retinervis and Xerophyta viscosa native to Madagascar which are flat bushes that develop magnificent violet flowers in the monsoon season.

One particular embodiment of the invention are preparations containing LEA proteins and/or LEA-like proteins and/or HS proteins which are obtained by extraction of yeasts and/or plants selected from the group consisting of yeasts and/or plants from the family of Saccharomyces, Pocacea, Scrophulariacea, Myrothamnacea, Velloziacea and/or the geni Boea, Ramonda, Hamelea, Chamaegigas, Selaginella and barley, wheat, corn, rice, peas, cowpeas, soya, onions, tomatoes, cotton, radishes, cucumbers and pineapples. Proteins according to the invention from Selaginella lepidophylla and survival fractions of protein-rich angiospermous or gymnospermous plants or microorganisms such as, for example, Saccharomyces cerevisiae are particularly suitable.

The LEA proteins are involved in the mechanisms of drought resistance in different ways. However, they all have the remarkable property of not denaturing, even in heat, and remain soluble even in boiling water. This property is attributable to high levels of hydrophilic amino acids. In one particular embodiment, the preparations according to the invention contain LEA proteins and/or LEA-like proteins and/or HS proteins which are distinguished by the fact that they do not denature in aqueous solution, even at temperatures of 100° C. Preferably at least 25, more preferably at least 40 and most preferably at least 50% by weight of the proteins consist of glutamic acid, glutamine and/or glycine. Of particular importance are the so-called LEA-D11 proteins which are also known as dehydrins. These species are distinguished by an amphiphilic structure, i.e. they possess both structural regions that are highly hydrophilic and others which are decidedly hydrophobic which gives them the ability to prevent the denaturing of a number of macromolecules. An accumulation of the dehydrins typically occurs when environmental stress causes dehydration, i.e. for example in the event of drought, high salt contents or frost. Apart from the fact that the family of the LEA proteins in general and the dehydrins in particular can be defined by their property of not denaturing in aqueous solution, even at temperatures of 100° C., they also have certain general structural features:

a so-called K segment which is an a-helical bipolar region that contains the following sequence of 15 amino acids: EKKGIMDKIKEKLPG;

a so-called S segment containing phosphorylatable serine residues;

and a so-called Y segment which is distinguished by an N-terminal sequence.

It appears that the LEA proteins from vegetable organisms have homologs in microorganisms such as, for example, Saccharomyces cerevisiae. Studies have shown that microorganisms contain proteins which have the same properties as or similar properties to the LEA proteins. The production of a protein with an amino acid sequence GAAKSKLNDA, which is known as HSP12 protein, was stimulated in Saccharomyces cerevisiae in particular by heating for 10 minutes to 80° C. By comparison with LEA proteins from plants, this 12 kDa large enzyme was actually identified as an LEA-like protein and not as HSP (Mtwisha et al.; Plant Mol. Biol., 1998; 37 (3), 513-512).

HS Proteins

Another constituent of the extracts or the survival fractions can be heat shock (HS) proteins. These are special proteins which are synthesized by cells and organisms at elevated temperature in response to heat shock (heat shock response). The increased formation of HS proteins leads in the skin to a transitional state in which the cells show improved resistance to further stress, for example in the form of UV radiation, oxidative stress, viral or bacterial infections, so that the danger of possibly irreparable cell damage is reduced. Table 1 below presents an overview by Trautinger et al. in which size, location and essential function are assigned to special HSPs.

TABLE 1
Heat shock proteins

| | Size | | |
|---|---|---|---|
| Name | [KD] | Location | Main Function |
| Ubiquitin | 8 | Cytosol/nucleus | Protein degradation |
| HSP 10 | 10 | Mitochondria | Co-factor of HSP 60 |
| HSP 27 | 27 | Cytosol/nucleus | Cell differentiation/ |
| | | | cell growth |
| Heme oxygenase-1 | 32 | Cytosol | Protection against |
| | | | oxidative damage |
| HSP 47 | 47 | Endoplasmic reticulum | Collagen companion |
| HSP 56 | 56 | Cytosol | Part of the steroid |
| | | | receptor complex |
| HSP 60 | 60 | Mitochondria | |
| HSP 72 | 70 | Cytosol/nucleus | |
| HSP 73 | 70 | Cytosol/nucleus | |
| Grp 75 | 70 | Mitochondria | |
| Grp 78 (Bip) | 70 | Endoplasmic reticulum | |
| HSP 90 | 90 | Cytosol/nucleus | Part of the steroid |
| | | | receptor complex |
| HSP 110 | 110 | Cytosol/nucleus | |

Heat shock proteins are a heterogeneous group of proteins with molecular weights in the range from 10 to 110 KD. They are mainly found in the cell core, in the mitochondriae and in the endoplasmic reticulum.

In another embodiment of the invention, the preparations additionally contain HS proteins, more particularly HSP 12 and/or HSP 70.

Under normal conditions, the HSPs perform important functions in the synthesis, transport and folding of proteins and, accordingly, are frequently referred to as “molecular companions”. Although their effect has not yet been fully understood, there is much evidence to suggest that the HSPs are added onto partly folded or misformed proteins and thus protect them against irreversible denaturing under stress [cf. Maytin JID, 104, 448 (1995)]. The two proteins HSP 27 and HSP 70 are of particular importance in this connection because they have particularly high heat tolerance, i.e. protect cells particularly effectively against further stress.

Extraction

The LEA proteins and/or LEA-like proteins and/or HS proteins are isolated by extraction of plants and/or microorganisms, more particularly yeasts, such as Saccharomyces cerevisiae, or resurrection plants. The extracts may be prepared in known manner, i.e. for example by aqueous, buffererd, alcoholic or aqueous/alcoholic extraction of the microorganisms, plants or plant parts. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found for example in Hagers Handbuch der pharmazeutischen Praxis (5th Edition, Vol. 2, pp. 1026-1030, Springer Verlag, Berlin-Heidelberg-N.Y. 1991). The percolation method is advantageous for industrial application. Fresh microorganisms, plants or plant parts are suitable as the starting material although dried or freeze-dried microorganisms, plants and/or plant parts which may be mechanically size-reduced before extraction are normally used. Any size reduction methods known to the expert, such as freeze grinding for example, may be used. Suitable solvents for the extraction process are organic solvents, water (preferably hot water with a temperature above 80° C. and, in particular, above 95° C.) or mixtures of organic solvents and water, more particularly low molecular weight alcohols with more or less large water contents. Extraction with methanol, ethanol, pentane, hexane, heptane, acetone, propylene glycols, polyethylene glycols and ethyl acetate, mixtures thereof and aqueous mixtures thereof is particularly preferred. The extraction process is generally carried out at 20 to 100° C., preferably at 30 to 90° C. and more particularly at 60 to 80° C. In one preferred embodiment, the extraction process is carried out in an inert gas atmosphere to avoid oxidation of the active principles of the extract. The extraction times are selected by the expert in dependence upon the starting material, the extraction process, the extraction temperature and the ratio of solvent to raw material, etc. After the extraction process, the crude extracts obtained may optionaly be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of freeze-dried microorganisms are in the range from 3 to 15 and more particularly 3 to 5% by weight. The present invention includes the observation that the extraction conditions and the yields of the final extracts may be selected by the expert according to the desired application. These extracts, which generally have active substance contents (=solids contents) of 0.5 to 10% by weight, may be used as such although the solvent may also be completely removed by drying, more particularly by spray drying or freeze drying.

Commercial Applications

The present invention also relates to the use of LEA proteins and/or LEA-like proteins, more particularly HSP12 and/or HS proteins, for the production of cosmetic and/or pharmaceutical preparations, in which they may be present in quantities of 0.01 to 10, preferably 0.1 to 1 and more partiularly 0.2 to 0.5% by weight, based on the preparation, and as active substances

for regulating the water metabolism in the skin or as moisturizers;

for strengthening the cell metabolism for protection against harmful environmental influences;

for protecting the skin and hair against free radicals and

for stimulating the synthesis of dermal macromolecules in the skin cells and cell membranes;

against ageing of the skin and as reactivating agents for the skin;

against damage to the skin by UV radiation.

Cosmetic and/or Pharmaceutical Preparations

The LEA proteins according to the invention may be used for the production of cosmetic and/or pharmaceutical preparations such as, for example, hair shampoos, hair lotions, foam baths, shower baths, creams, gels, lotions, alcoholic and aqueous/alcoholic solutions, emulsions, wax/fat compounds, stick preparations, powders or ointments. These preparations may also contain mild surfactants, oil components, emulsifiers, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, antidandruff agents, film formers, swelling agents, insect repellents, self-tanning agents, tyrosine inhibitors (depigmenting agents), hydrotropes, solubilizers, perservatives, perfume oils, dyes and the like as further auxiliaries and additives.

Surfactants Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysor-bates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols (cf. DE 197 56 377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, Dicaprylyl Carbonate (Cetiol® CC), Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Dicaprylyl Ether (Cetiol® OE), ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Emulsifiers

Suitable emulsifiers are, for example, nonionic surfactants from at least one of the following groups:

products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alk(en)yl group and ethoxylated analogs thereof;

addition products of 1 to 15 mol ethylene oxide onto castor oil and/or hydrogenated castor oil;

addition products of 15 to 60 mol ethylene oxide onto castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 mol ethylene oxide;

mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethyleneglycol-30 Dipolyhydroxystearate;

polymer emulsifiers, for example Pemulen types (TR-1, TR-2) of Goodrich;

polyalkylene glycols and

glycerol carbonate.

Ethylene Oxide Addition Products

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as lipid layer enhancers for cosmetic formulations from DE 20 24 051 PS.

Alkyl and/or Alkenyl Oligoglycosides

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide with the partial glycerides mentioned are also suitable.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide with the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerol-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 moles of ethylene oxide.

Anionic Emulsifiers

Typical anionic emulsifiers are aliphatic fatty acids containing 12 to 22 carbon atoms such as, for example, palmitic acid, stearic acid or behenic acid and dicarboxylic acids containing 12 to 22 carbon atoms such as, for example, azelaic acid or sebacic acid.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2 252 840 A and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacryl-amide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil. 108, 95 (1993).

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

UV Protection Factors and Antioxidants

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor as described in EP 0693471 B1;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably 4-methoxybenzalmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 197 12 033 A1 (BASF). The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used. Other suitable UV filters can be found in P. Finkel's review in SÖFW-Journal 122, 543 (1996) and in Parf. Kosm. 3, 11 (1999).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

Biogenic agents in the context of the invention are, for example, tocopherol, tocopherol acetate, tocopherol palmitate, ascorbic acid, deoxyribonucleic acid and fragmentation products thereof, β-glucans, retinol, bisabolol, allantoin, phytantriol, panthenol, AHA acids, amino acids, ceramides, pseudoceramides, essential oils, other plant extracts and vitamin complexes.

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Germ Inhibitors

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, famesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Enzyme Inhibitors

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Odor Absorbers

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of labdanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH regulators, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Insect Repellents

Suitable insect repellents are N,N-diethyl-m-toluamide, pentane-1,2-diol or Ethyl Butylacetylaminopropionate.

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipenta-erythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formal-dehyde solution, parabens, pentanediol or sorbic acid and the other classes of compounds listed in Appendix 6, Parts A and B of the Kosmetikverordnung (“Cosmetics Directive”).

Perfume Oils and Aromas

Suitable perfume oils are mixtures of natural and synthetic fragrances. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. These dyes are normally used in concentrations of 0.001 to 0.1% by weight, based on the mixture was a whole.

The total percentage content of auxiliaries and additives may be from 1 to 50% by weight and is preferably from 5 to 40% by weight, based on the particular preparation. The preparations may be produced by standard hot or cold processes and are preferably produced by the phase inversion temperature method.

### EXAMPLES

### Example 1

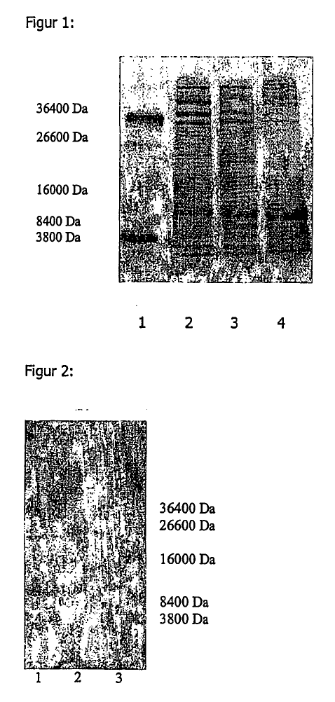

50 g freeze-dried Saccharomyces cerevisiae were suspended in 370 ml ammonium acetate buffer at 4° C. The buffer consisted of 50 mM ammonium acetate and 50 mM NaCl and had a pH of 7.5. The cells were destroyed with a sonificator at 4-10° C. During this procedure, the pH was kept at 7.5 with 2 n NaOH solution. The cell debris was removed by centrifuging at 4° C. and the supernatant solution was incubated for 10 mins. at 80° C. The solution was centrifuged once more and the 212 g of liquid obtained were spray-dried. The yield of dry matter was 4.7%, based on the quantity of yeast cells used. An SDS-PAGE analysis was carried out to determine the molecular weights of the proteins obtained. The results can be found in Example 4 and FIGS. 1-3.

### Example 2

4.5 liters of a medium containing 10 g/l of a bacteriological peptone, 20 g/l glucose, 5 g/l yeast extract and 0.8 M mannitol in osmotic water were autoclaved for 30 mins. at 121° C. 67 g freeze-dried Saccharomyces cerevisiae cells were added and cultivated under these osmotic stress conditions for 24 hours with shaking at 30° C. in the presence of air. The cells were harvested and centrifuged for 20 mins. at 4° C./5,600 G. The cells were then washed in ammonium acetate buffer (pH 7.5). 203 g biomass were obtained and 92 g of the biomass were suspended in 327 ml ammonium acetate buffer. The cells were destroyed with a sonificator at 4-10° C. During this procedure, the pH was kept at 7.5 with 2 n NaOH solution. The cell debris was removed by centrifuging at 4° C. and the supernatant solution was incubated for 10 mins. at 80° C. The solution was centrifuged once more and the 303 g of liquid obtained were spray-dried. The yield of dry matter was 3.0%, based on the quantity of yeast cells used. An SDS-PAGE analysis was carried out to determine the molecular weights of the proteins obtained. The results can be found in Example 4 and FIGS. 1-3.

### Example 3

1 kg fresh baker's yeast Saccharomyces cerevisiae was suspended in 2 liters water with 50 mM NaCl. The pH of the solution was adjusted to 7.5 with 2 n NaOH, after which the solution was heated for 15 mins. at 100° C. and then cooled. The cells were destroyed at 800 bar in a discontinuous high-pressure homogenizer. The concentration of the proteins obtained was 27 g/l. The pH was adjusted to 4 with 2 n sulfuric acid, after which the suspension was reheated for 15 mins. to 100° C. and then cooled. Insoluble fractions were removed by centrifuging for 30 mins. at 5600 G and the supernatant solution was filtered. The opalescent solution obtained was dried and 4.3% dry product were obtained. The opalescent solution obtained was dried and 4.3% dry product were obtained. The SDS-PAGE analysis of this extract can be found in Example 4 and FIGS. 1-3.

### Example 4

In order to test for the presence of LEA-like proteins HSP12 in the extracts of Examples 1 to 3, an anti-LEA-like protein HSP12 antiserum was prepared from synthetic peptide 1-15 MSDAGRKGFGEKASE-CONH2 and 95-109 YVSGRVHGEEDPTKK-CONH2 of the protein which was coupled to KLH proteins to obtain antigen. 3 mice were each immobilized 4 times with both antigens and the antiserum was obtained after 87 days.

The yeast extracts of Examples 1 to 3 were analyzed by SDS-PAGE analysis and colored with Coomassie Blue. The protein bands are shown in FIG. 1. The first trace is that of the comparison proteins while the other traces are those of the extracts of Examples 1-3.

1. Comparison proteins (36.4; 26.6; 16.0; 8.4 and 3.8 kDa)

2. Extract of Example 1 (30 mg/ml)

3. Extract of Example 2 (30 mg/ml)

4. Extract of Example 3 (30 mg/ml)

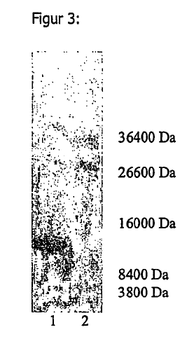

The immunoblot-SDS-PAGE analyses with anti-LEA-like protein HSP12 antiserum are shown in FIGS. 2 and 3.

FIG. 2: trace 1: extract of Example 2 (30 mg/ml)

trace 2: extract of Example 1 (30 mg/ml)

trace 3: comparison proteins (36.4; 26.6; 16.0; 8.4 and 3.8 kDa)

FIG. 3: trace 1: extract of Example 2 (30 mg/ml)

trace 2: comparison proteins (36.4; 26.6; 16.0; 8.4 and 3.8 kDa)

The results of the SDS-PAGE analyses both with Coomassie Blue and by immunoblot clearly show the presence of proteins which have a molecular weight of ca. 12 kDa.

### Example 5

In order to demonstrate the presence of HSP70 proteins, the extract of Example 3 was analyzed by SDS-PAGE electrophoresis with anti-human HSP70 antibody. FIG. 4 shows the SDS-PAGE. The antibody and human HSP70 were obtained from TEBU.

FIG. 4: trace 1: extract of Example 3 (30 mg/ml)

trace 2: human HSP70 (positive control)

trace 3: comparison proteins (216; 132; 78; 45.7; 32.5; 18.4 and 7.6 kDa)

The results of Example 4 and Example 5 demonstrate the presence of LEA-like HSP12 and of HSP70 proteins in the yeast extracts.

### Example 6

### Skin Moisture Regulating Test

Background: The epidermis of human skin contains the horny layer (the stratum corneum). The Stratum corneum is a dielectric medium of low electrical conductivity. The water content leads to an increase in the dielectrical conductivity so that determination of the dielectrical conductivity of the stratum corneum can serve as a measure of the moisture content of human skin. The increase in the dielectrical conductivity of the Stratum corneum reflects an increase in the moisture content of human skin.

Method: Samples of normal skin obtained from plastic surgery were used for this test. The Stratum corneum from these skin samples was stored in chambers with defined relative moisture (44%, saturated potassium carbonate solution) and standardized. Each sample of the Stratum corneum was comparatively tested under three conditions, namely:

1. without treatment

2. treatment with placebo

3. treatment with a preparation consisting of a hydrogel (Hyrogel LS from Laboratoire Sérobiologique LS) containing 4.3% by weight of extract of Example 3 (pH 6.14).

The placebo was the hydrogel (Hydrogel LS from Laboratoire Sérobiologioque) without the described preparation, i.e. without yeast extract. The hydrogel was applied in a quantity of 1 mg/cm².

The moisture-regulating activity of the above-described preparation was determined as a percentage increase in conductivity by comparison with the placebo treatment.

The results reflect a dose-dependent moisture-regulating activity.

TABLE 1
Moisture-regulating effect as determined by measurement of the
dielectrical conductivity (in μS); (the standard deviation is
shown in brackets)

| Type of | Before the | | | | | | |
|---|---|---|---|---|---|---|---|
| treatment | treatment | 30 mins. | 1 h | 2 h | 4 h | 6 h | 24 h |
| Control | 9.6 (1.2) | 9.9 | 10.0 | 10.3 | 9.9 | 9.7 | 9.5 |
| | | (1.2) | (1.0) | (1.1) | (1.0) | (1.2) | (1.1) |
| Placebo | 10.4 (1.0) | 28.4 | 16.3 | 12.1 | 11.5 | 10.3 | 9.1 |
| | | (2.2) | (1.2) | (1.3) | (0.8) | (0.8) | (1.0) |
| Treatment 3 | 10.1 (1.2) | 41.8 | 29.9 | 17.8 | 15.8 | 13.3 | 9.4 |
| | | 95.6) | (3.9) | (2.6) | (1.8) | (1.4) | (0.9) |

The control measurements show a uniform conductivity over the entire period of measurement. The placebo hydrogel produced a slight increase in the conductivity of the Stratum corneum after 30 mins. and after 1 hour. However, the moisture-regulating effect of the yeast extract is clearly reflected in the increase in conductivity 30 mins. and up to 6 hours after the treatment.

### Example 7

### Skin Regenerating and Revitalizing Activity

The object of this test is to demonstrate the regenerating and revitalizing activity of extracts of Saccharomyces cerevisiae on human fibroblast cultures in vitro.

Method 1: Effects on cell growth. Human fibroblasts were inoculated with 10% by weight of foetal calf serum in a defined nutrient medium (DMEM=Dulbecco Minimum Essential Medium, a product of Life Technologie S.a.r.I.) and incubated for 24 h at 37° C. in a 5% CO₂ atmosphere. The nutrient medium containing foetal calf serum was then replaced by a nutrient medium of DMEM without foetal calf serum. Active substance in the form of the extracts of Example 3 was then added to this nutrient medium in various concentrations. For comparison, a test series of human fibroblasts with no active substance was incubated as control. After the fibroblasts had been incubated for three days in the nutrient medium, growth and metabolic activity were evaluated by determining the protein content using Bradford's method (Bradford et al.; Anal. Biochem.; 1976; 72; 248-254) and the intracellular content of ATP using Vasseur's method (Journal Francais Hydrologie, 1981, 9, 149-156). The study shows that the extracts of Example 3 stimulate the growth and metabolism of the human fibroblasts in vitro to a considerable extent.

Method 2: improvement of viability. The test was carried out on human fibroblasts. It enables a certain number of parameters to be quantitatively determined on the resting cells. The cultivation of the cells corresponds to the cultivation of method 1 except for the incubation time. The incubation time for this test before the growth medium was replaced was 72 h. Viability was evaluated by colorimetric determination of the percentage protein content by Bradford's method (Anal. Biochem. 1976, 72, 248-254), by determination of the percentage glutathione content (GSH) with a fluorescent probe, orthophthaldehyde, by Hissin and Hilf's method (Anal. Biochem. 1976, 74, 214-216). The glutathione is produced by cells in order to be able to react directly against oxidative stress and environmental influences, such as high heavy metal levels. Accordingly, an increased percentage content of reduced glutathione after treatment of the cells with the extracts of Example 3 is a measure of the increased viability of the cells under the effect of external stress and other challenges. Besides the protein and glutathione contents, the DNA content was also determined with a bis-benzimide fluorescence probe (Hoechst). The results were expressed in percent by comparison with the control.

TABLE 3
Revitalizing activity (standard deviation in brackets)

| | Concentration (% by weight) | Protein content |
|---|---|---|
| Control | | 100 |
| Extract of Example 3 | 0.001 | 107 |
| | 0.003 | 124 |
| | 0.1 | 165 |
| | 0.3 | 173 |

TABLE 4

| | Concentration | DNA | Protein | GSH | ATP |
|---|---|---|---|---|---|
| | (% by weight) | content | content | content | content |
| Control | | 100 | 100 | 100 | 100 |
| Extract of | 0.1 | 127 | 134 | 117 | 144 |
| Ex. 3 | 0.3 | 136 | 169 | 114 | 177 |

The results show that extracts containing LEA-like HSP12 proteins from Saccharomyces cerevisiae stimulate the metabolism of human fibroblasts and thus show revitalizing and reactivating activity. They show growth-promoting activity and increase the viability of the fibroblasts. Accordingly, the extracts may be used in the cosmetics field in

antiageing preparations and/or as reactivating preparations

in preparations for increasing the resistance of human skin and hair follicles to harmful environmental influences

in preparations for stimulating the synthesis and renewal of dermal macromolecules, such as collagen, elastin, glycoproteins, proteoglycan and GAG.

### Example 8

### Anti-Inflammatory Properties In Vitro—UVB Protection

Background: UVB rays (280-320 nm) cause inflammation (erythema, edema) by activating an enzyme, namely phospholipase A2 or PLA2, which removes arachidonic acid from the phospholipids of the cell membrane. Arachidonic acid is the precursor of the prostaglandins which cause inflammation and cell membrane damage; the prostaglandins E2 (=PGE2) are formed by cyclooxygenase.

Method: The effect of UVB radiation was investigated in vitro in keratinocytes by determining the release of the cytoplasm enzyme LDH (lactate dehydrogenase). This enzymes serves as a marker for cell damage.

To carry out the tests, a defined medium containing foetal calf serum was inoculated with the keratinocytes and the extract of Example 3 was added 72 hours after the inoculation. The keratinocytes were then exposed to a dose of UVB (50 mJ/cm²—tubes: DUKE GL40E).

After incubation for another day at 37° C./5% CO₂, the LDH and PGE2 content in the supernatant was determined. The LDH (lactate dehydrogenase) content was determined by an enzyme reaction (kit used to determine LDH levels from Roche). The PGE2 content was determined by an ELISA test (ELISA kit from Roche). After the trypsin treatment, the cells were centrifuged and counted. The number of adhering keratinocytes was determined (after trypsin treatment) with a particle counter.

TABLE 5
Cell protecting effect of an extract of Saccharomyces cerevisiae
against UVB rays; results in % based on the control,
mean value of 2 tests each repeated twice

| | Concentration | Number of | Content | Content |
|---|---|---|---|---|
| | (% by weight) | keratinocytes | of released LDH | of released PGE2 |
| Control | | 100 | 0 | 0 |
| Control + UVB | | 37 | 100 | 100 |
| UVB + extract of | 0.1 | 45 | 71 | 105 |
| Example 3 | 0.3 | 42 | 59 | 80 |
| | 1 | 104 | 23 | 5 |

The results of these tests show that an extract according to the invention reduces the effect of UVB radiation on the number of keratinocytes. There is a reduction in the PGE2 content induced by UVB in human keratinocytes and a reduction in the content of released LDH in the cytoplasm. Accordingly, the described extracts have the ability to reduce cell membrane damage caused by UVB radiation and inhibit UVB-induced inflammation. Accordingly, the extracts may be used for the treatment of sensitive skin and for reducing damage by sunburn.

### Example 9

### Cell Protection Against Heat Shock in Human Fibroblasts

The heat shock in human fibroblasts was induced by increasing the incubation temperature from 37° C. to 45° C. for two hours. The number of living stressed cells was determined through the content of cellular adenosine triphosphate (ATP) and lactate dehydrogenase (LDH). The ATP content is well-known marker of cellular viability and a modified content is a very sensitive test for cytotoxicity. The content was determined by Vasseur's method (Vasseur P. et al.; Environmental Pollution; 1; 167-175; 1980).

The release of the high molecular weight cytoplasm enzyme LDH is a sign of cell membrane damage and is a general marker for cell damage. The LDH (lactate dehydrogenase) content was spectrophotometrically determined by determining the NADH content during the LDH-catalyzed conversion of pyruvate to lactate by Bonnekoh's method (Bonnekoh B. et al.; Dermatol. Research; 282; 325-329;1990).

Method: To carry out these tests, a defined culture medium (DMEM) containing the fibroblasts was inoculated with fetal calf serum and added to the plant extract or to the mixtures and preparations to be tested (in the defined medium containing 10% fetal calf serum) 72 hours after inoculation. Incubation was carried out at 37° C./5% CO₂.

After incubation for 48 hours at 37° C./5% CO₂, the cells were exposed to the heat shock by increasing the incubation temperature from 37° C. to 45° C. for two hours. The cells were then re-incubated for 24 hours at 37° C./5% CO₂.

The ATP content was monitored by determining the light component in the enzymatic reaction between ATP and the complex of luciferin/luciferase.

TABLE 6
Content of released LDH and released ATP for
determining the cell protecting effect against heat
shock (mean value of three tests each
repeated twice)

| | | | Content |
|---|---|---|---|
| | Concentration | ATP | of released |
| Treatment | (% by weight) | content [%] | LDH [%] |
| Control without | | 100 | 0 |
| heat shock (37° C.) | | | |
| Control with heat | | 37 | 100 |
| shock (45° C.) | | | |
| Extract of Example 3 + | 0.2 | 124 | 119 |
| heat shock | | | |
| | 0.6 | 188 | 98 |
| | 1 | 157 | 61 |

The harmful effect of heat shock on human fibroblasts was reflected in the reduced ATP content and the increased content of released LDH. The treatment with extract containing LEA-like HSP12 proteins resulted in cell resistance to heat shock.

TABLE 7
Cosmetic preparations (water, preservative to 100% by weight)

| Composition (INCI) | 1 | 2 | 3 | 4 | 5 | 6 |
|---|---|---|---|---|---|---|
| Emulgade ® SE | 5.0 | 5.0 | 5.0 | 4.0 | — | — |
| Glyceryl Stearate (and) Ceteareth 12/20 (and) Cetearyl Alcohol | | | | | | |
| (and) Cetyl Palmitate | | | | | | |
| Eumulgin ® B1 | — | — | — | 1.0 | — | — |
| Ceteareth-12 | | | | | | |
| Lameform ® TGI | — | — | — | — | 4.0 | — |
| Polyglyceryl-3 Isostearate | | | | | | |
| Dehymuls ® PGPH | — | — | — | — | — | 4.0 |
| Polyglyceryl-2 Dipolyhydroxystearate | | | | | | |
| Monomuls ® 90-O 18 | — | — | — | — | 2.0 | — |
| Glyceryl Oleate | | | | | | |
| Cetiol ® HE | — | — | — | — | — | 2.0 |
| PEG-& Glyceryl Cocoate | | | | | | |
| Cetiol ® OE | — | — | — | — | 5.0 | 6.0 |
| Dicaprylyl Ether | | | | | | |
| Cetiol ® PGL | — | — | — | 3.0 | 10.0 | 9.0 |
| Hexyldecanol (and) Hexyldecyl Laurate | | | | | | |
| Cetiol ® SN | 3.0 | 3.0 | 3.0 | — | — | — |
| Cetearyl Isononanoate | | | | | | |
| Cetiol ® V | 3.0 | 3.0 | 3.0 | — | — | — |
| Deyl Oleate | | | | | | |
| Myritol ® 318 | — | — | — | 3.0 | 5.0 | 5.0 |
| Coco Caprylate Caprate | | | | | | |
| Bees Wax | — | — | — | — | 7.0 | 5.0 |
| Nutrilan ® Elastin E20 | 2.0 | 2.0 | — | — | — | — |
| Hydrolyzed Elastin | | | | | | |
| Extract of Example 3 | 0.1 | 0.1 | 0.1 | 0.1 | 0.1 | 0.1 |
| Nutrilan ® I-50 | — | — | 2.0 | — | — | — |
| Hydrolyzed Collagen | | | | | | |
| Gluadin ® AGP | — | — | — | 0.5 | — | — |
| Hydrolyzed Wheat Gluten | | | | | | |
| Gluadin ® WK | — | — | — | — | 0.5 | 0.5 |
| Sodium Cocoyl Hydrolyzed Wheat Protein | | | | | | |
| Highcareen ® | 1.0 | 1.0 | 1.0 | 1.0 | 1.0 | 1.0 |
| Betaglucan | | | | | | |
| Hydagen ® CMF | 1.0 | 1.0 | 1.0 | 1.0 | 1.0 | 1.0 |
| Chitosan | | | | | | |
| Magnesium Sulfate Hepta Hydrate | — | — | — | — | 1.0 | 1.0 |
| Glycerol (86% by weight) | 3.0 | 3.0 | 3.0 | 5.0 | 5.0 | 3.0 |
| Composition (INCI) | 7 | 8 | 9 | 10 | 11 | 12 |
| Emulgade ® SE | 5.0 | 5.0 | 5.0 | 4.0 | — | — |
| Glyceryl Stearate (and) Ceteareth 12/20 (and) Cetearyl | | | | | | |
| Alcohol (and) Cetyl Palmitate | | | | | | |
| Eumulgin ® B1 | — | — | — | 1.0 | — | — |
| Ceteareth-12 | | | | | | |
| Lameform ® TGI | — | — | — | — | 4.0 | — |
| Polyglyceryl-3 Isostearate | | | | | | |
| Dehymuls ® PGPH | — | — | — | — | — | 4.0 |
| Polyglyceryl-2 Dipolyhydroxystearate | | | | | | |
| Monomuls ® 90-O 18 | — | — | — | — | 2.0 | — |
| Glyceryl Oleate | | | | | | |
| Cetiol ® HE | — | — | — | — | — | 2.0 |
| PEG-& Glyceryl Cocoate | | | | | | |
| Cetiol ® OE | — | — | — | — | 5.0 | 6.0 |
| Dicaprylyl Ether | | | | | | |
| Cetiol ® PGL | — | — | — | 3.0 | 10.0 | 9.0 |
| Hexyldecanol (and) Hexyldecyl Laurate | | | | | | |
| Cetiol ® SN | 3.0 | 3.0 | 3.0 | — | — | — |
| Cetearyl Isononanoate | | | | | | |
| Cetiol ® V | 3.0 | 3.0 | 3.0 | — | — | — |
| Deyl Oleate | | | | | | |
| Myritol ® 318 | — | — | — | 3.0 | 5.0 | 5.0 |
| Coca Caprylate Caprate | | | | | | |
| Bees Wax | — | — | — | — | 7.0 | 5.0 |
| Nutrilan ® Elastin E20 | 2.0 | 2.0 | — | — | — | — |
| Hydrolyzed Elastin | | | | | | |
| Extract of Example 3 | 0.1 | 0.1 | 0.1 | 0.1 | 0.1 | 0.1 |
| Nutrilan ® I-50 | — | — | 2.0 | — | — | — |
| Hydrolyzed Collagen | | | | | | |
| Gluadin ® AGP | — | — | — | 0.5 | — | — |
| Hydrolyzed Wheat Gluten | | | | | | |
| Gluadin ® WK | — | — | — | — | 0.5 | 0.5 |
| Sodium Cocoyl Hydrolyzed Wheat Protein | | | | | | |
| Highcareen ® | 1.0 | 1.0 | 1.0 | 1.0 | 1.0 | 1.0 |
| Betaglucan | | | | | | |
| Hydagen ® CMF | 1.0 | 1.0 | 1.0 | 1.0 | 1.0 | 1.0 |
| Chitosan | | | | | | |
| Magnesium Sulfate Hepta Hydrate | — | — | — | — | 1.0 | 1.0 |
| Glycerol (86% by weight) | 3.0 | 3.0 | 3.0 | 5.0 | 5.0 | 3.0 |

(1, 2) Soft cream,
(3, 4) moisturizing emulsion,
(5, 6) night cream
(7, 8) Soft cream,
(9, 10) moisturizing emulsion,
(11, 12) night cream

All the registered names in Table 7 designate products of the Cognis Group.

1. Cosmetic and/or pharmaceutical preparations containing late embryogenesis abundant (LEA) proteins and/or LEA-like proteins.

2. Preparations as claimed in claim 1, characterized in that they contain LEA proteins and/or LEA-like proteins which are obtained by extraction of yeasts and/or plants selected from the group consisting of yeasts and/or plants from the family of Saccharomyces, Pocacea, Scrophulariacea, Myrothamnacea, Velloziacea and/or the geni Boea, Ramonda, Hamelea, Chamaegigas, Selaginella and barley, wheat, corn, rice, peas, cowpeas, soya, onions, tomatoes, cotton, raddishes, cucumbers and pineapples.

3. Preparations as claimed in claims 1 and/or 2, characterized in that they contain LEA proteins and/or LEA-like proteins which are distinguished by the fact that they do not denature in aqueous solution, even at temperatures of 100° C.

4. Preparations as claimed in at least one of claims 1 to 3, characterized in that the LEA-like proteins are HSP12 proteins from Saccharomyces cerevisiae.

5. Preparations as claimed in at least one of claims 1 to 4, characterized in that they contain LEA proteins and/or LEA-like proteins which are distinguished by the fact that at least 25% by weight consists of the amino acids glutamic acid, glutamine and/or glycine.

6. Preparations as claimed in at least one of claims 1 to 5, characterized in that they contain 0.001 to 10% by weight, based on the quantity of solids and the final preparation, of the LEA proteins and/or LEA-like proteins.

7. Preparations as claimed in claim 1, characterized in that they additionally contain heat shock proteins (HS proteins).

6. The use of LEA proteins and/or LEA-like proteins and/or HS proteins for the production of cosmetic and/or pharmaceutical preparations.

7. The use of LEA proteins and/or LEA-like proteins and/or HS proteins as active substances for regulating the water metabolism in the skin.

8. The use of extracts of LEA proteins and/or LEA-like proteins and/or HS proteins as active substances for strengthening the cell metabolism for protection against harmful environmental influences.

9. The use of LEA proteins and/or LEA-like proteins and/or HS proteins as active substances for protecting the skin and hair against free radicals.

10. The use of LEA proteins and/or LEA-like proteins and/or HS proteins as active substances for stimulating the synthesis of dermal macromolecules in the skin cells and cell membranes.

11. The use of LEA proteins and/or LEA-like proteins and/or HS proteins as active substances against ageing of the skin and as skin reactivating agents.

12. The use of LEA proteins and/or LEA-like proteins and/or HS proteins as active substances against damage to the skin by UV radiation.
